(19) United States
(12) Patent Application Publication
(10) Pub. No.: US 2019/0262528 A1
(43) Pub. Date: Aug. 29, 2019

(54) DEVICE FOR REMOVING NOXAE FROM BLOOD, EXTRACORPOREAL PERFUSION SYSTEM COMPRISING SUCH A DEVICE AND METHOD OF MANUFACTURING SUCH A DEVICE

(71) Applicant: B. BRAUN AVITUM AG, MELSUNGEN (DE)

(72) Inventor: PETER MANDRY, Dresden (DE)

(21) Appl. No.: 16/277,187

(22) Filed: Feb. 15, 2019

(30) Foreign Application Priority Data
Feb. 23, 2018 (DE) 102018104177.2

(51) Int. Cl. A61M 1/34; A61M 1/16; A61M 1/36

(52) U.S. Cl. CPC A61M 1/3496; A61M 1/16; A61M 1/3621; A61M 1/3672; A61M 1/3431; A61M 2205/3334; A61M 1/3437

(57) ABSTRACT

A device for removing noxae from blood, in an extracorporeal perfusion system, includes a housing and a plurality of hollow fibers provided inside the housing, which can be perfused by the blood. The hollow fibers each have a plurality of pores that permit the plasma of the blood to flow through the pores from an inside of the hollow fibers to an outside of the hollow fibers. The hollow fibers are modified or pretreated, in particular chemically, in such a way that they have a functionalized surface which binds the noxae to itself and removes the noxae from the blood. An inside surface of the hollow fibers includes a coating, in particular a hemocompatible and anticoagulant coating, which is arranged to prevent damage to the cellular components of the blood when the blood flows through the hollow fibers. An extracorporeal perfusion system includes the device.

### RELATED APPLICATION

This application claims the benefit of priority of German Patent Application No. 10 2018 104 177.2, filed Feb. 23, 2018, the content of which is incorporated by reference herein in its entirety.

### FIELD

The present disclosure relates to a device for removing, in particular negatively charged, noxae from blood, which comprises plasma and cellular components, in an extracorporeal perfusion system, comprising a housing and a plurality of hollow fibers provided within the housing, which are configured to be perfused by blood, the hollow fibers each having a plurality of pores which are formed such that the plasma of the blood can flow through the pores from an inside of the hollow fibers to an outside of the hollow fibers, the hollow fibers being modified or pretreated, in particular chemically, such that they have a functionalized surface which binds the noxae to itself and removes them from the blood. Furthermore, the present disclosure relates to an extracorporeal perfusion system comprising such a device and a method for manufacturing such a device.

### BACKGROUND

Sepsis or blood poisoning is a complex systemic inflammatory reaction of the human organism caused by infection by bacteria, their toxins or fungi. With many patients, severe sepsis or septic shock is still fatal despite all therapeutic measures. Reasons for the occurrence of sepsis include the use of catheters and endoscopes, the implantation of prostheses, surgical interventions, the use of immunosuppressive drugs, the increase in older patients and the increasing resistance of bacteria to antibiotics. Today, infections of patients are often caused by (multi-)resistant bacteria.

Prior art methods already known for some time such as plasmapheresis, also referred to as plasma exchange treatment, or antibody therapy methods have not been able to significantly improve the prognosis of septic patients. In particular, plasmapheresis has turned out to be non-selective and inefficient, since in addition to toxins and pro-inflammatory cytokines, also protective, anti-inflammatory mediators are withdrawn from the patient. Furthermore, a single therapy cycle requires an exchange volume of about 12 liters of plasma (about 50 donors), which entails an additional risk of infections or allergic reactions. Antibody therapy methods are very expensive due to the technical complexity of obtaining, purifying and characterizing the antibodies in question and their use poses a risk of an allergic counter-reaction of the body to the antibodies.

Furthermore, prior art also knows the treatment of blood or plasma in an extracorporeal perfusion system for the neutralization or elimination of pathogenic blood components such as lipopolysaccharides, lipoteichonic acids etc. using suitable adsorber materials.

For example, U.S. Pat. No. 4,576,928 or DE 39 32 971 describe porous carrier materials with immobilized polymyxin B, which, however, have proved unsuitable for clinical application, since the polymyxin B ligand causes severe nephrotoxic and neurotoxic damage when released into the bloodstream.

In DE 41 13 602 A1 polyethyleneimine-modified percelluloses are disclosed as adsorbers, which however have a low binding capacity for lipopolysaccharides, so that when used in an extracorporeal perfusion system the medically tolerable extracorporeal dead volume is exceeded.

DE 44 35 612 A1 also describes a plasma perfusion method which is suitable for the elimination of lipopolysaccharides and TNF-α (tumor necrosis factor α). However, this method is very complex, hemodynamically disadvantageous, since it requires a very large extracorporeal dead volume, and apart from lipopolysaccharides and TNF-α also eliminates fibrinogen which is essential for plasmatic coagulation, so that application of this method is limited to only two to three consecutive treatments, depending on the initial concentration of fibrinogen, which is usually insufficient for effective treatment of a patient.

A particularly effective removal of negatively charged noxae from blood plasma is disclosed in EP 1 602 387 A1. In the device disclosed in this publication, hollow fibers are provided which are chemically modified such that the charged lipopolysaccharides (LPS) and lymphotoxin α (LTA) are particularly well bound to them and can thus be removed from the plasma. Here, the hollow fibers are chemically modified at the surface, preferably by graft polymerization. In graft polymerization, compounds, such as anion exchangers (groups), with good binding properties for LPS and LTA are grafted onto the hollow fiber material. The anion exchangers are longer chains in the design of tentacles with a plurality of cationic groups. Such tentacle-like extensions on the hollow fiber base material are capable of binding several LPS or LTA molecules, thus allowing to increase the efficiency of the hollow fibers. Synthetic, semi-synthetic or natural polycation chains, which can be present in linear or branched form, are preferably used for the modification of the hollow fibers by tentacles. The hollow fibers are preferably modified by (poly) cation chains which contain tertiary or quaternary amines.

However, the device disclosed in EP 1 602 387 A1 has the disadvantage that the chemically modified, coated or grafted surface is incompatible with blood cells and the cellular components of the blood, so that the blood cells must be separated from the blood plasma by plasma separation prior to treatment. Commercially available plasma separators consist of hollow fiber capillaries with a pore size of 0.1 to 0.5 μm. They are used for a maximum period of 4 to 6 hours. Since a patient suffering from sepsis is treated for a period of at least 74 hours, the plasma separator must therefore be changed very frequently during this period.

Document EP 1 776 175 B1 discloses a continuous method for the production of a regioselective, porous hollow fiber membrane, where the hollow fiber membrane thus produced allows blood separation and blood purification in one step. The hollow fiber membrane is basically made of a blood compatible polymer and therefore does not damage the cellular components of the blood. Only the outside of the hollow fiber membrane and the pores are equipped with functional groups via a special plasma treatment. When blood is finally passed through the hollow fibers at high pressure, only (blood) plasma penetrates the fine pores. The cellular blood components are too large and remain in the blood-compatible main channel. Finally, in the fine pores and the outer wall of the hollow fibers, grafted binding molecules fish the toxins out of the fluid via wet-chemical treatment.

However, the method disclosed in EP 1 776 175 B1 has the disadvantage that it requires a complex vacuum system with a plurality of vacuum chambers for plasma pretreatment of the hollow fiber membrane/the hollow fibers, making the production of the hollow fiber membrane disclosed therein very complex.

### SUMMARY

Against this background, it is the object of the present disclosure to avoid or at least mitigate the disadvantages of the prior art and in particular to provide blood separation and blood purification in one step (without prior plasma separation) with porous hollow fibers produced in a simpler manner than in prior art/with a device for removing noxae from blood produced in a simpler manner than in prior art. In particular, a simple treatment system with long-lasting application time should be provided.

This object is achieved by a device for removing noxae from blood, an extracorporeal perfusion system, and a method for manufacturing a device for removing noxae from blood. Advantageous embodiments and further developments are explained below.

The present disclosure relates firstly to a device for removing, in particular negatively charged, noxae from blood, which comprises plasma and cellular components, in/for/for use in an extracorporeal perfusion system, comprising a housing and a plurality of hollow fibers/hollow fiber capillaries provided within the housing, which are configured to be perfused by blood, the hollow fibers each having a plurality of pores which are formed such that the plasma of the blood can flow through the pores from an inside of the hollow fibers to an outside of the hollow fibers, the hollow fibers being modified or pretreated, in particular chemically, such that they have a functionalized surface which binds the noxae to itself and removes them from the blood, wherein, preferably exclusively, an inside surface of the hollow fibers is further provided (completely/the entire inside surface) with a cover/coating being in particular hemocompatible and anticoagulant and arranged to prevent/avoid damage to the cellular components of the blood when the blood flows through the hollow fibers.

A noxae in the context of this application is a material or substance which is present in an undesirable manner in the blood of a living being, for example a human being, and has a harmful, pathogenic and/or endangering effect on the organism or a body organ. Noxae can be understood as lipopolysaccharides (LPS, endotoxins), lipoteichonic acids (LTA), viruses, DNA, etc. An extracorporeal perfusion system is a circulatory system outside the body of the living being. If blood is spoken of in the context of this application, a suspension of plasma and cellular components such as erythrocytes, leukocytes, thrombocytes, etc. is to be understood.

The device according to the present disclosure is designed in such a manner that both the cellular components and the plasma of the blood flow through it, so that no separation of the plasma from the cellular components is necessary before the blood flows through the device. Therefore, no plasma separation is required and hence no frequent change of a plasma separator is necessary according to the present disclosure. The device of the present disclosure is used to treat patients with diseases caused by an invasion of gram-negative and/or gram-positive bacteria or other negatively charged noxae such as shigatoxin.

Full reference is made to EP 1 602 387 A1 with regard to the material of the porous hollow fibers/hollow fibers comprising pores and with respect to the modification/pre-treatment of the hollow fibers. However, the most important aspects are also briefly outlined below in the present application.

In principle, hollow fiber materials can be used which are made of polyamide, polysulfone, polyether, polyethylene, polypropylene, polyester or derivatives and/or mixtures of such polymers. Hollow fibers are particularly preferably made of nylon (polyamide 66). These membrane base materials can be modified by methods known per se, preferably by graft polymerization, to give them a functionalized surface that binds the noxae to itself and removes them from the blood. In the context of the present application, a functionalized surface is distinguished by a large surface area and functional groups attracting noxae, thus promoting both a mechanical and a specific adhesion of the noxae to the hollow fibers. In particular, the hollow fibers employed in the device according to the present disclosure are chemically modified in such a way that negatively charged noxae such as LPS or LTA molecules can bind particularly well to the hollow fiber material and are thus removed from the blood (hollow fibers with positive charge).

A chemical modification of the hollow fiber material is therefore preferably carried out by graft polymerization, in which compounds are grafted onto the hollow fiber material which show good binding properties, especially for LPS and/or LTA. It has been shown to be particularly advantageous to graft anion exchange groups onto the hollow fiber material. Such anion exchange groups are preferably designed as longer chains with a multitude of cationic groups, as so-called tentacles. Such tentacle-like extensions on the base material are capable of binding several LPS or LTA molecules. Synthetic and/or semi-synthetic and/or natural polycation chains are preferably used for the modification of the hollow fiber material by means of tentacles, whereby these chains can be present in linear or branched form. It is particularly preferred that the hollow fiber materials according to the present disclosure are modified by cation or polycation chains which contain tertiary and/or quaternary amines.

Preferred anion exchanger groups on the hollow fiber materials include di- or trialkylaminoalkyl, di- or trialkylaminoaryl, di- or triarylaminoalkyl, di- or triarylaminoaryl, di- or trialkylammoniumalkyl, di- or triarylammoniumalkyl, di- or triarylammoniumaryl and di- or trialkylammoniumaryl radicals. Furthermore, polymers of positively charged amino acids or amino acids containing tertiary or quaternary amino groups such as polylysine, polyarginine or polyhistidine or copolymers or derivatives thereof are suitable as anion exchange materials within the scope of the present disclosure, as is polyethyleneimine. The device particularly preferably comprises a polyamide-based hollow fiber material modified with diethylaminoalkyl or diethylaminoaryl radicals, in particular diethylaminoethyl polyamide.

The multitude of the hollow fibers/hollow fiber capillaries forms a hollow fiber membrane.

The housing of the device according to the present disclosure can be seen as a membrane module, which has a hollow fiber membrane/a multitude of porous hollow fibers inside. The device according to the present disclosure is thus similar to a dialyzer with blood caps and side port. The pores of the hollow fibers have a size of about 0.1 to 0.5 μm, so that only the plasma of the blood can flow through the pores, but not the cellular components/blood cells. The hollow fibers/the hollow fiber membranes have a large inside surface.

The core of the present disclosure is that only the inside surfaces of the modified hollow fibers, which come into contact with the cellular components of the blood when it flows through the hollow fibers, are coated or covered with a coating/covering that does not damage the cellular components of the blood. Thus, according to the present disclosure, there is no functionalized surface on the inside surfaces of the hollow fibers that binds the noxae to itself and removes them from the blood. Only the pores and outside surfaces of the hollow fibers thus have the functionalized surface which binds the noxae to itself and removes them from the blood. This is why the cellular components can flow through the hollow fibers without being damaged. The noxae are removed from the plasma when the plasma flows through the pores or along the outside surfaces of the hollow fibers. The device of the present disclosure thus provides a regioselective membrane adsorber.

The coating is therefore preferably arranged or formed on the inside surface of the hollow fibers in such a way that the inner circumferential sides of the pores are not covered or incompletely covered by the coating.

In an advantageous way, the coating on the inside surface of the hollow fibers is both hemocompatible and anticoagulant as well as compatible with the functionalized surface of the hollow fibers to which the coating is applied on the inside of the hollow fibers and which binds the noxae to itself and removes them from the blood. In particular, the coating/covering is compatible with the cellular components of the blood so that they are not damaged as they flow through the device. In addition, the coating or the substance formed by the coating is compatible with the functionalized original (inside) surface of the hollow fibers that binds the noxae to itself and removes them from the blood, so that the coating has good adhesion thereon. In other words, the hemo-incompatible surface of the hollow fibers/hollow fiber membrane on the blood side is covered with a hemocompatible substance/coating that is both compatible with the hemo-incompatible surface and very well tolerated by the blood.

A preferred exemplary embodiment is characterized in that the coating is applied to the inside surface of the hollow fibers by making a solution flow through the hollow fibers. In other words, the solution/substance on the blood side flows through the hollow fiber membrane/the hollow fibers. The functionalized surface of the hollow fibers is preferentially de-functionalized on the inside of the hollow fibers, i.e. saturated or bound by the solution. Preferably, the solution is or contains an anticoagulant substance which binds to the inside surface of the hollow fibers.

More preferably, the solution is a negatively charged anionic solution, in particular an anticoagulant polyanion, preferably heparin. In this way, it can be achieved that the basically positively charged, functionalized surface of the hollow fibers is saturated/neutralized/discharged by the negatively charged solution. In other words, damage to the blood cells in the present disclosure is avoided/prevented precisely by the fact that the functional groups on the inside surface of the hollow fibers are bonded/saturated by the solution and at the same time an anticoagulant substance such as heparin binds to the inside surface of the hollow fibers.

In a preferred exemplary embodiment of the present disclosure, the coating is an anionic coating. However, other coatings such as cationic or hydrophilic coatings are also conceivable according to the present disclosure.

Preferably, a coating of only the inside surfaces of the hollow fibers and a saturation of the inside surfaces of the hollow fibers with the (anionic) solution as well as a variation of the thickness of the coating can be achieved by adjusting a quantity, a flow rate and preferably an anion concentration of the (anionic) solution.

It has been found that in particular the parameters quantity/liquid amount/volume of the (anionic) solution which is introduced into the device as well as the flow rate of the (anionic) solution at which the solution flows through the device or the hollow fibers must be suitably adjusted. If an anionic solution is used, the parameter of the anion concentration also has a large influence and must be set appropriately. The quantity/liquid amount/volume of the solution has a particular effect on the saturation of the inside surfaces with the solution and on the thickness or layer thickness of the coating or covering that can be achieved on the inside surface. In other words, the thickness of the covering/coating can be controlled/adjusted by the quantity of the solution/substance flowing through the hollow fibers (quantity control). The thickness of the coating is preferably adjusted in such a way that only a small part of the existing surface area and thus of the available capacity is lost. The flow rate and the anion concentration have a particular effect on the fact that only the inside surfaces of the hollow fibers are coated, but not the pores and outside surfaces of the hollow fibers. It should be noted at this point that the active surface for the removal of the noxae is essentially located in the pores/in the membrane. The effective surface in the pores is preferably over 1500 times larger (e.g. about 1600 times larger) than the inside surface or the outside surface of the hollow fiber. Against this background, the inactivation of the inside surface of the hollow fiber results in only a negligible loss of capacity.

The housing is preferably closed on the outlet side, especially via a valve, when the solution enters the hollow fibers. If blood flows through the device, the housing is open on the outlet side, so that the device is basically not operated in the so-called dead-end method during the treatment of a patient.

In other words, in order to achieve hemocompatibility with (full) blood, the outlet of the hollow fibers is first closed and a negatively charged solution flows through the inside of the hollow fibers, so that the positively charged groups on the inside of the hollow fibers are saturated with the negatively charged solution, but the pores of the hollow fibers are not. This can be adjusted by the quantity/flow rate/concentration of the solution flowing therethrough. During the treatment of a patient, the ends of the hollow fibers/hollow fiber capillaries are open and blood flows through the hollow fibers. The bound functional groups and the bound anticoagulant substance on the inside of the hollow fibers prevent damage to the blood cells.

The coating can preferably be re-dosed during the treatment of a patient.

Further preferred, the device has a tangential filter design so that both ends of the housing are open.

The device can also be used as a plasma filter for long-term applications.

Furthermore, the present disclosure relates to an extracorporeal perfusion system comprising a device for the removal of noxae from blood as described above. In particular, the extracorporeal perfusion system further has a pump which conveys the plasma of the blood out of the hollow fibers at least partially via the pores and, downstream of the device, returns it to the blood which has flowed through the device.

Since the device is arranged to be perfused by both the cellular components and the plasma of the blood, so that no separation of the plasma from the cellular components is required before the blood flows through the hollow fibers, no plasma separation/no plasma separator is required in the extracorporeal perfusion system of the present disclosure.

The present disclosure also relates to a method for manufacturing a device for removing noxae from blood, in particular a device as described above, comprising the steps of: a) manufacturing a plurality of porous hollow fibers, preferably of plastic, further preferred of polyamide, polysulfone, polyether, polypropylene, polyester or derivatives and/or mixtures of such polymers; b) modifying or pretreating the hollow fibers, preferably chemically, more preferably by graft polymerization, in such a way that they have a functionalized surface which binds the noxae to itself and removes them from the blood; c) inserting of the plurality of porous hollow fibers into a housing; and d) causing a flow of a preferably negatively charged, anionic solution, in particular an anticoagulant polyanion, preferably heparin, through the plurality of porous hollow fibers located in the housing (before the start of treatment); wherein the method steps a) to d) are carried out in chronological order, i.e. first a), then b), then c) and finally d).

The method according to the present disclosure is particularly suitable for a modification of an inner coating of hollow fibers/hollow fiber capillaries.

It is preferred that the method also comprises the step e) of adjusting a quantity and a flow rate of the solution; wherein method step e) is carried out before method step d).

Further preferably, the method also comprises the step f) of closing the housing on the outlet side, in particular via a valve, before the solution enters the hollow fibers.

It should be noted that, with regard to the characteristics of the method according to the present disclosure, full reference is still made to the previous explanations concerning the device according to the present disclosure and the extracorporeal perfusion system according to the present disclosure. Furthermore, full reference is made to EP 1 602 387 A1 with regard to the methods steps a) and b).

### BRIEF DESCRIPTION OF THE DRAWING FIGURES

The present disclosure is further explained below with the help of Figures wherein:

FIG. 1 shows a schematic view of an extracorporeal perfusion system according to the present disclosure;

FIG. 2 shows a perspective view of a device according to the present disclosure for the removal of noxae from blood;

FIG. 3 shows a perspective side view of the device according to the present disclosure;

FIG. 4 shows a schematic sectional view of the device according to the present disclosure;

FIG. 5 shows a perspective view of a hollow fiber provided in the device;

FIG. 6 shows a schematic view of the hollow fiber;

FIG. 7 shows a schematic sectional view of the hollow fiber in which a blood treatment known from prior art is illustrated;

FIG. 8 shows a schematic sectional view of the hollow fiber in which a blood treatment according to the present disclosure is illustrated; and

FIG. 9 shows a flowchart of the method according to the present disclosure.

The Figures are merely schematic in nature and serve exclusively to understand the present disclosure. Identical elements are provided with the same reference signs.

### DETAILED DESCRIPTION

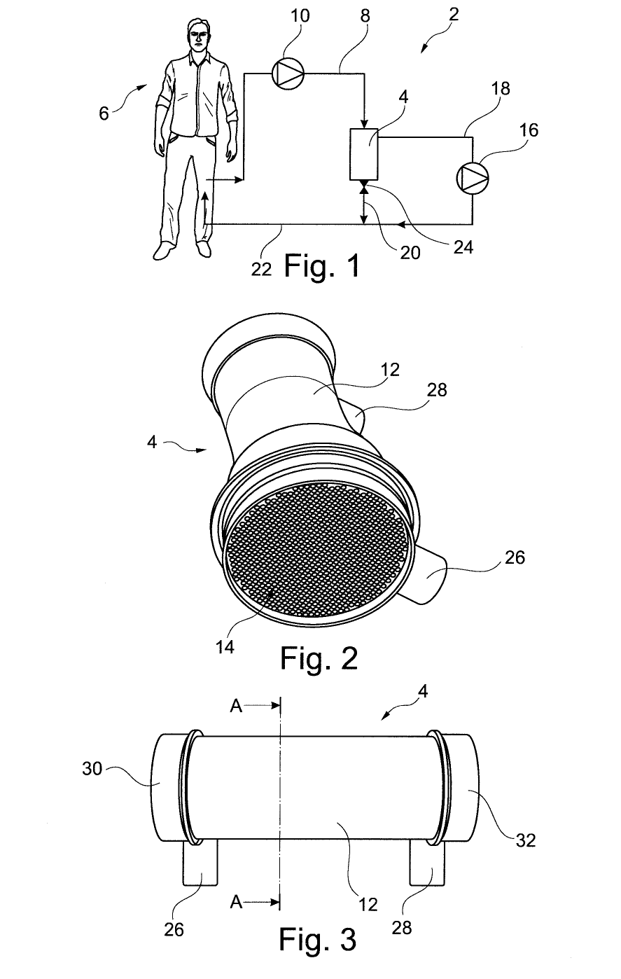

FIG. 1 shows a schematic view of an extracorporeal perfusion system 2 according to the present disclosure comprising a device 4 for the removal of noxae from blood. In this method, blood is taken from a human 6, which is pumped by means of a first pump 10 via a first line 8 to the device 4. The device 4 comprises, as shown in FIG. 2, a housing 12 and a multitude of hollow fibers 14 located inside the housing 12. The blood is essentially supplied to the hollow fibers 14. The hollow fibers 14 are porous, so that the plasma of the blood (blood plasma) can at least partly be sucked/pumped by means of a second pump 16 out of the device 4 and into a second line 18. At least the cellular components of the blood, such as erythrocytes, leukocytes or thrombocytes, leave the device 4 via a third line 20. Downstream of the device 4, the second line 18 and the third line 20 converge again and the blood is returned to the human 6 via a fourth line 22. In the extracorporeal perfusion system 2 according to the present disclosure, the blood with all its components, i.e. in particular both with plasma and blood cells, is fed to the device 4. No separate plasma separator is required to separate the plasma from the blood cells. The device 4 is designed to clean the blood and remove noxae from the blood. A shut-off valve 24 is provided on the outlet side of the device 4 at the beginning of the third line 20.

FIG. 2 shows a perspective view of the device 4 according to the present disclosure, comprising a housing 12 and a plurality of hollow fibers 14 located within the housing 12. The housing 12 has an essentially tube-like/tubular/cylindrical shape. A first port 26 is provided on the outer peripheral surface of the housing 12 near an inlet side of the housing 12 and a second port 28 is provided near an outlet side of the housing 12. The second line 18 shown in FIG. 1 can be connected to the first port 26 and/or to the second port 28 in order to convey the plasma of the blood out of the device 4 by means of the second pump 16.

FIG. 3 shows a perspective side view of the device 4 according to the present disclosure. In the view shown in FIG. 3, the device 4 is covered on the inlet side by a first cover cap 30 and on the outlet side by a second cover cap 32. The cover caps 30, 32 are of identical design and are adapted in shape and size to the round/circular inlet or outlet of the housing 12.

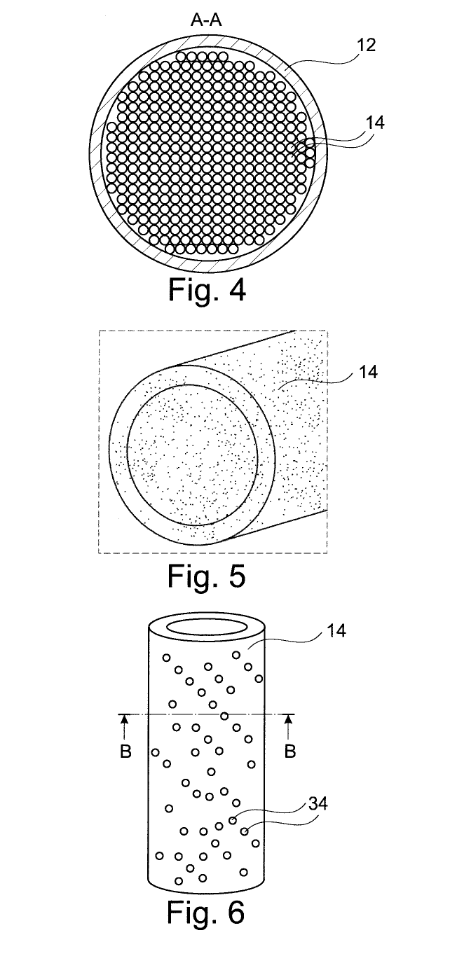

FIG. 4 shows a schematic sectional view of the device 4 according to the present disclosure, taken along the section line A-A shown in FIG. 3. In the view shown in FIG. 4, the hollow fibers 14 are shown slightly enlarged in order to illustrate the arrangement of the hollow fibers 14 within the housing 12 better than is the case in FIG. 2. The plurality of hollow fibers 14 extend in the longitudinal/axial direction of the substantially tubular/cylindrical housing 12 and fill substantially the entire interior space defined by the housing 12 (see also FIG. 2). The entirety of the hollow fibers 14 fauns a hollow fiber membrane.

FIG. 5 shows an enlarged perspective view of a single hollow fiber 14 provided in the device 4. The base material of the hollow fiber 14 is preferably polyamide on which diethylaminoalkyl or diethylaminoaryl is grafted in tentacular fashion (not shown). As indicated in FIG. 5, the hollow fibers 14 are porous.

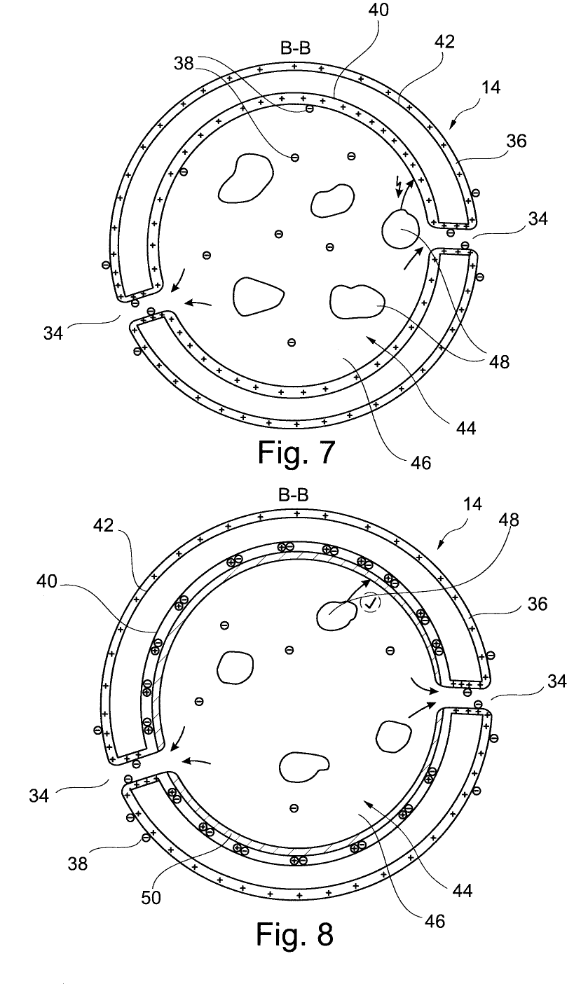

FIG. 6 shows a schematic view of the hollow fiber 14, in which the porous structure is represented by a plurality of enlarged pores 34. FIG. 7 and FIG. 8 are sectional views of the hollow fiber 14 shown in FIG. 6, taken at the section line B-B shown in FIG. 6.

The core aspects of this present disclosure are explained using FIG. 7 and FIG. 8. FIG. 7 illustrates a blood treatment known from the prior art of EP 1 602 387 A1 and FIG. 8 shows a blood treatment according to the present disclosure.

The hollow fiber 14 shown in FIG. 7 has a functionalized surface 36. The functionalized surface 36 is positively charged and is designed to bind noxae 38 to itself which are found in the blood 44 and to remove them from the blood 44. The functionalized surface 36 is provided both on an inside surface 40 of the hollow fiber 14 and an outside surface 42 of the hollow fiber 14 as well as in the area of the pores 34. The functionalized surface 36 is produced by a chemical modification, in particular by graft polymerization.

If now blood 44, which has blood plasma 46 and blood cells 48, flows through the hollow fiber 14 shown in FIG. 7, the negatively charged noxae 38 located in the blood 44 and in particular in the blood plasma 46 are bound to the positively charged (surface of the) hollow fiber 14 both on the inside surface 40 and outside surface 42 as well as in the area of the pores 34 and are thus removed from the blood. The size or diameter of the pores 34 is such that the blood cells 48 cannot flow through the pores 34. If the blood cells 48 shown in FIG. 7 come into contact with the functionalized surface 36, the blood cells 48 are damaged/destroyed, as indicated by a flash in FIG. 7. In the prior art, the blood cells 48 must therefore be separated from the blood plasma 46 so that the blood cells 48 do not enter the device 4 or the hollow fibers 14.

According to the present disclosure, the inside surface 40 of the hollow fibers 14 is further provided with a hemocompatible and anticoagulant coating 50 (see FIG. 8). The coating 50 is applied by causing a flow of a negatively charged anionic solution (e.g. an anticoagulant polyanion such as heparin) through the hollow fibers 14 (before the blood treatment shown). This ensures that, on the one hand, the functionalized, positively charged surface 36 on the inside surface 40 of the hollow fibers 14 is bound/discharged/neutralized by the negatively charged anionic solution, as illustrated in FIG. 8 by the contiguous positive and negative, hence neutralizing charges on the inside surface 40 of the hollow fibers 14. On the other hand, a coating 50 binds to the (previously) functionalized surface 36. The coating 50 is hemocompatible and anticoagulant, so that the blood cells 48 flowing through the hollow fibers 14 are not damaged when they hit the inside surface 40 (indicated by a checkmark in FIG. 8). The coating 50 is compatible with the functionalized surface 36 and adheres to it.

If now blood 44, which contains blood plasma 46 and blood cells 48, flows through the hollow fiber 14 shown in FIG. 8, the negatively charged noxae 38 located in the blood 44 and in particular in the blood plasma 46 are only bound to the outside surface 42 and in the region of the pores 34 to the positively charged (surface of the) hollow fiber 14 and thus removed from the blood. No noxae 38 are bound to the inside surface 40 of the hollow fiber 14 and, as already explained, the blood cells 48 are thus not damaged. In the device 4 according to the present disclosure, it is therefore not necessary to separate the blood cells 48 from the blood plasma 46 upstream of the device 4.

By setting a flow rate and an anion concentration of the anionic solution which flows through the hollow fiber 14 before the blood treatment shown, it is possible to coat only the inside surfaces 40 of the hollow fibers 14, but not the pores 34 and the outside surfaces 42 of the hollow fibers 14. This is achieved in particular by setting the flow rate to a low value and the anion concentration to a high value (more viscous anionic solution). By adjusting the quantity of the anionic solution, a saturation of the inside surfaces 40 of the hollow fibers 14 and a thickness of the coating 50 can be adjusted.

Here it applies that the coating 50 becomes the thicker the larger the quantity/amount of liquid is which enters the hollow fibers 14.

The shut-off valve 24 shown in FIG. 1 is closed when the anionic solution is introduced into the device 4 or the multitude of hollow fibers 14.

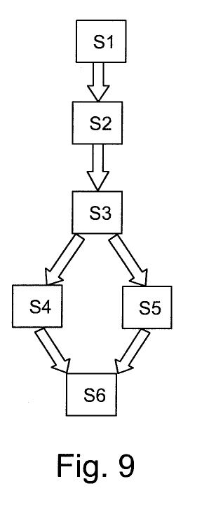

FIG. 9 illustrates a flow chart of the method according to the present disclosure. In accordance with the method according to the present disclosure, a plurality of porous hollow fibers 14 is first produced in step S1. The hollow fibers 14 are then modified/pretreated in step S2 in such a way that they have a functionalized surface 36 which binds the noxae to itself and removes them from the blood. Then, in step S3, the plurality of porous hollow fibers 14 is inserted into a housing 12. In step S4, the housing 12 is closed on the outlet side by a valve (the shut-off valve 24) and, in parallel, a quantity, a flow rate and an anion concentration of an anionic solution are adjusted in step S5. Finally, in step S6, the anionic solution is caused to flow through the hollow fibers 14 located in the housing 12.

1. A device for removing noxae from blood which comprises plasma and cellular components in an extracorporeal perfusion system, comprising:
a housing and a plurality of hollow fibers provided inside the housing and configured to be perfused by the blood, wherein
the hollow fibers each have a plurality of pores configured such that the plasma of the blood can flow through the pores from an inside of the hollow fibers to an outside of the hollow fibers, and wherein
the hollow fibers are modified or pretreated in such a way that they have a functionalized surface which binds the noxae to itself and removes the noxae from the blood, wherein
an inside surface of the hollow fibers is further provided with a coating which is configured to prevent damage of the cellular components of the blood when the blood flows through the hollow fibers.

2. The device according to claim 1, wherein the coating is arranged or formed on the inside surface of the hollow fibers in such a way that the inner circumferential sides of the pores are not covered or incompletely covered by the coating.

3. The device according to claim 1, wherein the coating on the inside surface of the hollow fibers is both hemocompatible and anticoagulant as well as compatible with the functionalized surface of the hollow fibers which binds the noxae to itself and removes them from the blood, and to which the coating is applied on the inside surface of the hollow fibers.

4. The device according to claim 1, wherein the coating is applied to the inside surface of the hollow fibers by causing a solution to flow through the hollow fibers.

5. The device according to claim 4, wherein the solution is a negatively charged anionic solution.

6. The device according to claim 4, wherein the coating is only applied to the inside surface of the hollow fibers and a saturation of the inside surface of the hollow fibers with the solution is achieved, and a thickness of the coating is varied by adjusting a quantity, a flow rate and an anion concentration of the solution.

7. The device according to claim 4, wherein the housing is closed on an outlet side when the solution is introduced into the hollow fibers.

8. An extracorporeal perfusion system comprising a device for removing noxae from blood according to claim 1 and a pump which conveys the plasma of the blood out of the hollow fibers via the pores and feeds it back downstream of the device to the blood which has flowed through the device.

9. A method for producing a device for removing noxae from blood comprising the steps:
a) producing a plurality of porous hollow fibers;
b) modifying or pretreating the hollow fibers in such a way that they have a functionalized surface which binds the noxae to itself and removes the noxae from the blood;
c) inserting said plurality of porous hollow fibers into a housing; and
d) causing a solution to flow through the plurality of porous hollow fibers located in the housing;
the method steps a) to d) being carried out in chronological order.

10. The method according to claim 9, further comprising the step:
e) adjusting a quantity and a flow rate of the solution; wherein method step e) is carried out before method step d).

11. The method according to claim 9, further comprising the step:
f) closing the housing on an outlet side before the solution is introduced into the hollow fibers.
